(19) United States
(12) Patent Application Publication
(10) Pub. No.: US 2026/0034330 A1
(43) Pub. Date: Feb. 5, 2026

(54) NEURAL REHABILITATION SYSTEMS AND METHOD

(71) Applicant: National Central University, Taoyuan City (TW)

(72) Inventors: Po-Lei Lee, Taoyuan City (TW); Po-Yi Tsai, Taoyuan City (TW)

(73) Assignee: National Central University, Taoyuan City (TW)

(21) Appl. No.: 18/931,739

(22) Filed: Oct. 30, 2024

(30) Foreign Application Priority Data
Jul. 31, 2024 (TW) 113128442

(51) Int. Cl. A61M 21/00

(52) U.S. Cl. CPC A61M 21/00; A61M 2021/0027; A61M 2021/005

(57) ABSTRACT

A neural rehabilitation system and method is provided. The neural rehabilitation system comprises a prompting device, a brainwave measuring device, a brainwave recognition device, and a neural stimulation device. The prompting device is used to generate media messages. The media messages are used to induce mirror neurons in a brain, causing the brain to generate mirror neuron brainwave signals accordingly. The brainwave measuring device is suitable for being worn on the head to measure these mirror neuron brainwave signals. The brainwave recognition device generates trigger signals based on the mirror neuron brainwave signals. The neural stimulation device produces neural stimulation signals according to the trigger signals and delivers them to the brain and/or a body.

### CROSS REFERENCE TO RELATED APPLICATION

This application claims the priority of Taiwan Patent Application No. 113128442, filed on Jul. 31, 2024, entitled “Neural rehabilitation system and method”, and the disclosure of which is incorporated herein by reference.

### FIELD OF INVENTION

The present disclosure relates to a rehabilitation system and method, and more particularly, to a neural rehabilitation system and method.

### BACKGROUND OF INVENTION

Current neural rehabilitation methods include exercise therapy, functional electrical stimulation (FES), transcranial magnetic stimulation (TMS), etc. These technologies facilitate the recovery of patients' nervous function to varying extents, but each has limitations.

Exercise therapy is a way of physical therapy that teaches patients to do some movements. It improves or restore body functions through systematic exercise and activities. This therapy requires the active participation and continuous practice of the patients. However, many patients are always unable to successfully perform the required rehabilitation movements, leading to compromised results. Functional electrical stimulation (FES) is primarily used to restore muscle and motor function for patients with stroke or spinal cord injury. It works by stimulating specific muscles or nerves with electrical currents to promote muscle contraction. However, the effectiveness of FES is limited to superficial muscles and is less effective for patients with deep nerve injuries.

Transcranial magnetic stimulation (TMS) is a non-invasive method that stimulates specific regions of the brain by placing electromagnetic coils on the scalp to generate a magnetic field. Patents related to transcranial magnetic stimulation include U.S. Pat. Nos. 11,717,686B2, 11,478,603B2 and 8,958,882B1. Transcranial magnetic stimulation has been proven to have a certain level of effectiveness in treating depression and promoting neuroplasticity. However, the effectiveness of this treatment varies significantly among different patients.

### SUMMARY OF INVENTION

Research has shown that providing neural stimulation while the patient performs or imagines actions will more significantly enhance neurological recovery. Therefore, in light of the research and to overcome the limitations of prior arts, the present disclosure proposes a neural rehabilitation system and method utilizing a brainwave-based human-machine interface device. This system and method may guide the patient to imagine certain actions while simultaneously applying stimulation to the patient's body or brain through non-invasive instruments, effectively promoting neural function recovery.

The present disclosure provides a neural rehabilitation system, comprising: a prompting device for generating a media message, wherein the media message is used to induce mirror neurons of a brain, causing the brain to correspondingly generate a mirror neuron brainwave signal; a brainwave measuring device, suitable for being worn on the head, for measuring the mirror neuron brainwave signal; a brainwave recognition device for generating a trigger signal based on the mirror neuron brainwave signal; a neural stimulation device for generating a neural stimulation signal based on the trigger signal and delivering the neural stimulation signal to the brain and/or a body.

In one embodiment of the present disclosure, the prompting device includes a screen, AR/VR glasses, or speakers, and the media message includes images, videos, or sounds.

In one embodiment of the present disclosure, the brainwave measuring device includes a wearable electroencephalograph machine and a functional near-infrared spectroscopy device.

In one embodiment of the present disclosure, the brainwave recognition device utilizes a machine learning classifier or deep learning network to determine whether the mirror neuron brainwave signal is available.

In one embodiment of the present disclosure, the neural stimulation device includes a transcranial magnetic stimulator, a transcranial electrical stimulator, and a muscle stimulation device.

The present disclosure provides a neural rehabilitation method, comprising: generating a media message by a prompting device, wherein the media message is used to induce mirror neurons of a brain, causing the brain to correspondingly generate a mirror neuron brainwave signal; measuring the mirror neuron brainwave signal by a brainwave measuring device; generating a trigger signal based on the mirror neuron brainwave signal by a brainwave recognition device; generating a neural stimulation signal based on the trigger signal and delivering the neural stimulation signal to the brain and/or a body by a neural stimulation device.

In one embodiment of the present disclosure, the prompting device includes a screen, AR/VR glasses, or speakers, and the media message includes images, videos, or sounds.

In one embodiment of the present disclosure, the brainwave measuring device includes a wearable electroencephalograph machine and a functional near-infrared spectroscopy device.

In one embodiment of the present disclosure, the brainwave recognition device utilizes a machine learning classifier or deep learning network to determine whether the mirror neuron brainwave signal is available.

In one embodiment of the present disclosure, the neural stimulation device includes a transcranial magnetic stimulator, a transcranial electrical stimulator, and a muscle stimulation device.

### DESCRIPTION OF DRAWINGS

FIG. 1 is a schematic diagram of a neural rehabilitation system of the present disclosure.

FIG. 2 is a flowchart of a neural rehabilitation method of the present disclosure.

### DETAILED DESCRIPTION OF PREFERRED EMBODIMENTS

In order to make the above and other objectives, features, and advantages of the present disclosure more obvious and understandable, the following exemplifies the preferred embodiments of the present disclosure, combined with the accompanying drawings, and describe in detail as follows.

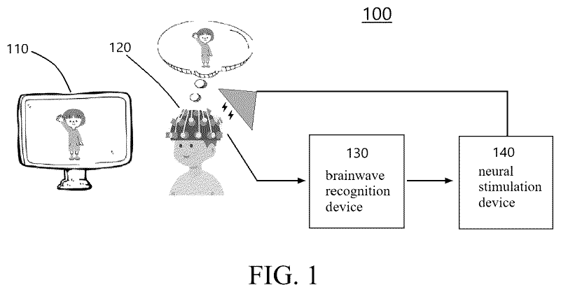

The figures in the subject application are illustrative. Specifically, the appearance of the components in FIG. 1 is schematic and not intended to limit the scope of the present disclosure. For example, the prompting device 110 in FIG. 1 is roughly depicted as a screen to facilitate reader's understanding. Additionally, for simplicity, only a block and a triangle are used to represent the structure of neural stimulation device 140, which may include transcranial magnetic stimulators, muscle stimulation devices, etc.

Firstly, refer to FIG. 1. FIG. 1 is a schematic diagram of a neural rehabilitation system of the present disclosure. In this embodiment, the neural rehabilitation system 100 includes a prompting device 110, a brainwave measuring device 120, a brainwave recognition device 130, and a neural stimulation device 140. The prompting device 110 can generate media messages, including images, animations, videos, sounds, etc. The prompting device 110 may include a screen, AR/VR glasses, or a speaker, or combinations thereof. The media messages generated by the prompting device 110 are generally used to convey an instruction, such as the action of raising the right hand, allowing users (patients) to imagine themselves performing this instruction in their mind. If a user needs to receive instructions visually, a screen displaying images, animations, or videos can be selected as the prompting device 110. If the user can only receive instructions auditorily, a speaker producing sounds can be selected as the prompting device 110. When the user truly receives the instructions conveyed by the media messages, the mirror neurons in the user's brain will be instinctively induced, leading the brain to correspondingly generate brainwave signals. The brainwave signals generated during this process will hereinafter be referred to as mirror neuron brainwave signals to distinguish them from brainwave signals produced by other neural activities in the brain.

The brainwave measuring device 120 is typically worn on the user's head and can measure various brainwave signals, including the mirror neuron brainwave signals targeted by the present disclosure. The brainwave measuring device 120 includes equipment such as a wearable brainwave instrument and functional near-infrared spectroscopy, to accurately capture the user's brain activities. These devices are capable of real-time monitoring and recording of activities in different brain regions, providing high-resolution brainwave data.

The brainwave recognition device 130 is used to identify the types of brainwave signals and verify whether the brainwave signals are usable. Since the brainwave measuring device 120 may continuously and consistently measure various brainwave signals (which may or may not include mirror neuron brainwave signals), it is necessary by the brainwave recognition device 130 to further verify whether usable mirror neuron brainwave signals are present. Specifically, the brainwave recognition device 130 first identifies whether the types of the brainwave signals measured by the brainwave measuring device 120 include mirror neuron brainwave signals. If so, the brainwave recognition device 130 further verifies whether the mirror neuron brainwave signals are usable. It should be noted that, hereinafter, the process of detecting the mirror neuron brainwaves and verifying whether they are usable is referred to as “determining whether the mirror neuron brainwave signals are available.”

In some cases, the user may not focus on receiving the media messages from the prompting device 110 and instead think of other things, causing the type of brainwave signals generated by the brain is not a mirror neuron brainwave signal. In other cases, the user may incorrectly receive the media message from the prompting device 110 and mistakenly imagine an action that is not indicated. For example, the media message may deliver an instruction of “raising the right hand,” but the user misunderstands it as “raising the left hand.” As a result, the mirror neuron brainwave signal generated by the user's brain corresponds to “raising the left hand,” rather than the mirror neuron brainwave signal that should correspond to “raising the right hand” as instructed by the media message. At this point, the brainwave recognition device 130 will consider the mirror neuron brainwave signal corresponding to “raising the left hand” as unusable.

To effectively and accurately determine whether mirror neuron brainwave signals are available, in this embodiment, the brainwave recognition device 130 utilizes a machine learning classifier or deep learning network to collect and establish each user's brainwave models, accumulating large amounts of data to train the brainwave recognition device 130. The data cover different users' brainwave patterns under various scenarios. Through continuous learning and optimization, the system can more accurately recognize and classify mirror neuron brainwave signals. The accuracy of the brainwave recognition device 130 in determining the availability of mirror neuron brainwave signals is enhanced through this process.

After verifying that the mirror neuron brainwave signals are usable, the brainwave recognition device 130 will generate trigger signals based on the usable mirror neuron brainwave signals and transmits them to the neural stimulation device 140. In other words, if usable mirror neuron brainwave signals are not present, trigger signals will not be generated. This process involves precisely interpreting and converting brainwave signals to ensure the accuracy and effectiveness of subsequent neural stimulation. Finally, the neural stimulation device 140 generates neural stimulation signals based on the trigger signals and delivers them to the user's brain and/or body. In this embodiment, the neural stimulation device 140 may include devices such as a transcranial magnetic stimulator, transcranial electrical stimulator, or muscle stimulation device. For example, in the case of a transcranial magnetic stimulator, the neural stimulation signals, such as magnetic field strength and frequency, can be automatically adjusted based on the received trigger signals to ensure optimal stimulation. Similarly, the transcranial electrical stimulator can adjust the current intensity and duration according to the trigger signals.

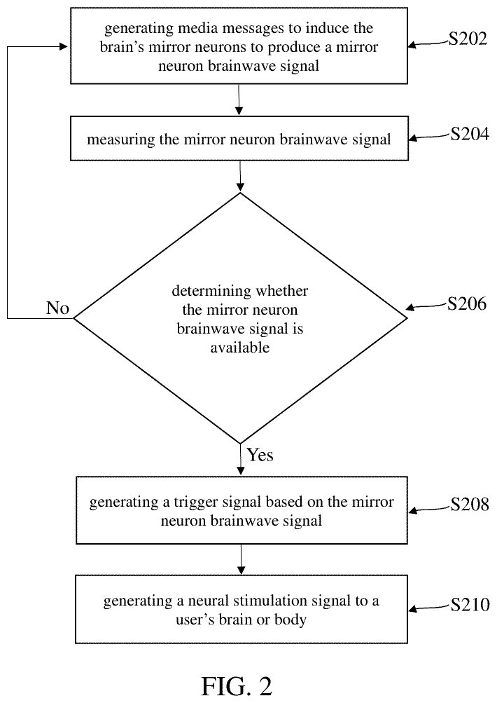

Please refer to FIG. 2. FIG. 2 is a flowchart of the neural rehabilitation method of the present disclosure. The neural rehabilitation method of the present disclosure includes steps S202 to S210. Firstly, in step S202: generating media messages to induce the brain's mirror neurons to produce a mirror neuron brainwave signal. In this embodiment, the media messages are generated by the prompting device 110. The media messages can include images, animations, videos, and sounds, among others. The prompting device 110 may include a screen, AR/VR glasses, or speakers, or combinations thereof. The media messages are generally used to convey an instruction, such as the action of raising the right hand, allowing users (patients) to imagine themselves performing this instruction in their mind. If a user needs to receive instructions visually, the prompting device 110 can display media messages, such as images, animations, or videos. If the user can only receive instructions auditorily, the prompting device 110 can produces sounds. When the user receives the instructions conveyed by the media messages, the brain's mirror neurons are instinctively induced, and a brainwave signal is generated correspondingly. This brainwave signal induced by the mirror neurons is referred to as a mirror neuron brainwave signal.

Step S204: measuring the mirror neuron brainwave signal. In this embodiment, the mirror neuron brainwave signal is measured by the brainwave measuring device 120. The brainwave measuring device 120 is typically worn on the user's head and can measure various brainwave signals, including the mirror neuron brainwaves targeted by the present disclosure. The brainwave measuring device 120 includes equipment such as a wearable brainwave instrument and a functional near-infrared spectroscopy, to accurately capture the user's brain activities. These devices are capable of real-time monitoring and recording of activities in different brain regions, providing high-resolution brainwave data.

Step S206: determining whether the mirror neuron brainwave signal is available. In this embodiment, the brainwave recognition device 130 identifies the types of brainwave signals and determines whether the brainwave signals are usable. Since the brainwave measuring device 120 may continuously and consistently measure various brainwave signals (which may include a mirror neuron brainwave signal), it is necessary to further verify usable mirror neuron brainwave signals by the brainwave recognition device 130. Specifically, the step includes identifying whether the types of the brainwave signals measured by the brainwave measuring device 120 include mirror neuron brainwave signals, and confirming whether the mirror neuron brainwave signals are usable. In some cases, the user may not focus on the media messages. If the user thinks of other things, the type of the brainwave signals generated by the brain may not be a mirror neuron brainwave signal. In other cases, the user may incorrectly receive the media message from the prompting device 110 and mistakenly imagine an action that is not indicated. For example, the media message may deliver an instruction of “raising the right hand,” but the user misunderstands it as “raising the left hand.” As a result, the mirror neuron brainwave signal generated by the user's brain corresponds to “raising the left hand,” rather than the mirror neuron brainwave signal that should correspond to “raising the right hand” as instructed by the media message. At this point, the brainwave recognition device 130 will consider the mirror neuron brainwave signal corresponding to “raising the left hand” as unusable. In other words, the step of “determining whether the mirror neuron brainwave signal is available” is the process of detecting a mirror neuron brainwave signal and confirming whether it is usable.

To effectively and accurately determine whether mirror neuron brainwave signals are available, in this embodiment, the brainwave recognition device 130 utilizes a machine learning classifier or deep learning network to collect and establish each user's brainwave models, accumulating large amounts of data to train the brainwave recognition device 130. The data cover different users' brainwave patterns under various scenarios. Through continuous learning and optimization, the system can more accurately recognize and classify mirror neuron brainwave signals. The accuracy of the brainwave recognition device 130 in determining the availability of mirror neuron brainwave signals is enhanced through this process.

Step S208: generating a trigger signal based on the mirror neuron brainwave signal. In this embodiment, after confirming that the mirror neuron brainwave signal is usable, the brainwave recognition device 130 will generates a trigger signal based on the usable mirror neuron brainwave signal. That is, if the usable mirror neuron brainwave signal is not present, the trigger signal will not be generated. This process involves accurately interpreting and converting the brainwave signals to ensure the precision and effectiveness of the subsequent neural stimulation.

Finally, step S210: generating a neural stimulation signal to a user's brain or body. In this embodiment, after the trigger signal is generated, a neural stimulation signal is produced based on the trigger signal and the neural stimulation signal is transmitted to the user's brain and/or body (including nerves and muscles) by the neural stimulation device 140. The neural stimulation device 140 may include devices such as a transcranial magnetic stimulator, transcranial electrical stimulator, or muscle stimulation device. For example, in the case of a transcranial magnetic stimulator, the neural stimulation signal, such as magnetic field strength and frequency, can be automatically adjusted based on the received trigger signal to ensure optimal stimulation. Similarly, the transcranial electrical stimulator can adjust the current intensity and duration according to the trigger signal.

A key advantage of the present disclosure is to implement neural entrainment. The precise neural modulation is achieved as the mirror neurons are induced by the prompting device 110 of the neural rehabilitation system 100, with the brainwave recognition device 130 and neural stimulation device 140 adjusting stimulation intensity and modes in real-time based on brain activities. This approach, which involves observing brainwave types and using instruments to stimulate the brain/body, allows for accurate rehabilitation of nerves and muscles in damaged areas of the brain/body. This not only enhances the specificity and effectiveness of treatment but also helps shorten recovery time, offering a more intelligent neurorehabilitation solution. Furthermore, the present disclosure employs machine learning classifiers or deep learning networks to flexibly adjust treatment plans, collect and analyze brainwave data of individual cases in real-time, and make real-time adjustments to better accommodate diverse treatment needs of individuals.

The above is only exemplary, rather than restrictive. Any equivalent modifications or changes without departing from the spirit and scope of the present disclosure should fall within the scope of the appended claims.

What is claimed is:

1. A neural rehabilitation system, comprising:
a prompting device for generating a media message, wherein the media message is used to induce mirror neurons of a brain, causing the brain to correspondingly generate a mirror neuron brainwave signal;
a brainwave measuring device, suitable for being worn on the head, for measuring the mirror neuron brainwave signal;
a brainwave recognition device for generating a trigger signal based on the mirror neuron brainwave signal;
a neural stimulation device for generating a neural stimulation signal based on the trigger signal and delivering the neural stimulation signal to the brain and/or a body.

2. The neural rehabilitation system according to claim 1, wherein the prompting device includes a screen, AR/VR glasses, or speakers, and the media message includes images, videos, or sounds.

3. The neural rehabilitation system according to claim 1, wherein the brainwave measuring device includes a wearable electroencephalograph machine and a functional near-infrared spectroscopy device.

4. The neural rehabilitation system according to claim 1, wherein the brainwave recognition device utilizes a machine learning classifier or deep learning network to determine whether the mirror neuron brainwave signal is available.

5. The neural rehabilitation system according to claim 1, wherein the neural stimulation device includes a transcranial magnetic stimulator, a transcranial electrical stimulator, and a muscle stimulation device.

6. A neural rehabilitation method, comprising:
generating a media message by a prompting device, wherein the media message is used to induce mirror neurons of a brain, causing the brain to correspondingly generate a mirror neuron brainwave signal;
measuring the mirror neuron brainwave signal by a brainwave measuring device;
generating a trigger signal based on the mirror neuron brainwave signal by a brainwave recognition device;
generating a neural stimulation signal based on the trigger signal and delivering the neural stimulation signal to the brain and/or a body by a neural stimulation device.

7. The method according to claim 6, wherein the prompting device includes a screen, AR/VR glasses, or speakers, and the media message includes images, videos, or sounds.

8. The method according to claim 6, wherein the brainwave measuring device includes a wearable electroencephalograph machine and a functional near-infrared spectroscopy device.

9. The method according to claim 6, wherein the brainwave recognition device utilizes a machine learning classifier or deep learning network to determine whether the mirror neuron brainwave signal is available.

10. The method according to claim 6, wherein the neural stimulation device includes a transcranial magnetic stimulator, a transcranial electrical stimulator, and a muscle stimulation device.
